(19) United States
(12) Patent Application Publication
(10) Pub. No.: US 2020/0367730 A1
(43) Pub. Date: Nov. 26, 2020

(54) DISPOSABLE ENDOSCOPE DEVICE

(71) Applicant: Nizam M. MEAH, Manvel, TX (US)

(72) Inventor: Nizam M. MEAH, Manvel, TX (US)

(21) Appl. No.: 16/775,626

(22) Filed: Jan. 29, 2020

Related U.S. Application Data
(62) Division of application No. 16/422,149, filed on May 24, 2019.

(51) Int. Cl. A61B 1/00; A61B 1/01; A61B 1/015; A61B 1/273; A61B 1/05

(52) U.S. Cl. CPC A61B 1/00135; A61B 1/01; A61B 1/015; A61B 1/273; A61B 1/05; A61B 1/00045; A61B 1/00103; A61B 1/00165; A61B 1/00009

(57) ABSTRACT

The present disclosure provides an endoscope device, which includes an optical tube including a proximal end connectable to a light source and the distal end is insertable into an internal organ of a subject. The optical tube is configured to transmit light from the proximal end to the distal end for illuminating the internal organ. A portion of a peripheral surface of the optical tube is configured with a first profile along a longitudinal direction. A disposable flexible tube is connectable to the optical tube and insertable into the internal organ along with the optical tube. The disposable flexible tube includes channels for administering a medical treatment to the subject. A portion of an outer surface of the flexible tube is configured with a second profile. The second profile is configured complementary to the first profile and engages with first profile, for coupling the flexible tube with the optical tube.

### TECHNICAL FIELD

The present disclosure relates to an endoscope device and, more particularly, relates to the endoscope device with a disposable flexible tube, which can be replaced after each use and resterilization of the endoscope.

### BACKGROUND

Endoscopy or use of an endoscope device for providing medical treatment to patients has been proliferated in recent years, mainly due to its non-invasive nature. As such, the patients subjected to the endoscopic procedures suffer negligible trauma, recuperate rapidly and experience minimal discomfort, which are typically associated with conventional surgical or medical procedures.

The endoscope device is typically an elongated tubular member, which is insertable into a hollow internal organ of the patient via a cavity, for enabling a medical practitioner to diagnose the internal organ. The elongated tubular member includes sophisticated optics for illuminating the internal organ and channels to route medical fluids into the internal organs. Typically, the endoscope device is selected based on the internal organ that is required to be diagnosed. As such, different types of endoscope devices are available based on the internal organ that is required to be diagnosed. As an example, for diagnosing internal organs such as but not limited to esophagus, stomach, and duodenum upper endoscope devices are employed. Similarly, a colonoscope is employed for diagnosing a colon of the patient, angioscopes for diagnosing blood vessels, bronchoscopes for examining bronchi and laparoscopes for examining the peritoneal cavity. Due to such versatility and complex construction of the endoscope device, they are generally expensive. For such expenses to be justified, these endoscopes must be reused with other patients.

To enable reusability, the endoscope device is sterilized or at least disinfected after each use with the patient, prior to use with a subsequent patient. One of the techniques employed for sterilizing, is the immersion of the endoscope into a disinfectant solution for a specific period of time, while also flushing the channels with the disinfectant solution. However, due to long channels in the endoscope, clean up of biological debris and bacteria is a hassle and incomplete at best. Also, due to the urgency of reuse, the endoscope may not be immersed within the disinfectant solution for the specific period of time nor the channels are flushed. This leads to formation of a bacterial biofilm over the endoscope, which makes the devices vulnerable to infections. Additionally, skilled personnel may be required for thoroughly sterilizing the endoscope using disinfectant solution, which adds on costs to the already expensive endoscope.

Another technique to sterilize the endoscope device is heat sterilization. In heat sterilization, the endoscope device is placed in an autoclave and subjected to heat for a predetermined time. However, the optics and electronics of the endoscope may malfunction during use when subjected to the heat sterilization, due to dysfunction of sophisticated parts within the endoscope, which is undesirable.

Therefore, there is a need for techniques which can overcome one or more limitations stated above in addition to providing other technical advantages.

### SUMMARY

Various embodiments of the present disclosure provide an endoscope device. The endoscope device includes an optical tube including a proximal end and a distal end, the proximal end is connectable to a light source and the distal end is insertable into an internal organ of a subject via a cavity. The optical tube is configured to transmit light from the proximal end to the distal end for illuminating the internal organ of the subject. A portion of a peripheral surface of the optical tube is configured with a first profile along a longitudinal direction. A disposable flexible tube is connectable to the optical tube and at least partially insertable into the internal organ along with the optical tube. The disposable flexible tube includes one or more channels for administering a medical treatment to the subject. A portion of an outer surface of the disposable flexible tube is configured with a second profile. The second profile is configured complementary to the first profile and adapted to engage with the first profile, for coupling the disposable flexible tube with the optical tube.

In another embodiment of the present disclosure, the endoscope device is disclosed. The device includes the optical tube including the proximal end connectable to the light source via a joystick and a distal end insertable into the internal organ of the subject via the cavity. The optical tube is configured to be a closed tube and includes at least one light-transmitting element for transmitting light from the proximal end to the distal end. A portion of the peripheral surface of the optical tube is configured with the first profile along a longitudinal direction. The first profile is configured to be an arcuate profile extending outwardly from an axis of the optical tube. At least one guide lens is configured at the distal end of the optical tube and located adjacent to the light-transmitting element for improving illumination. An image sensor is positioned to the distal end of the optical tube and communicably coupled to a display device, wherein the image sensor is configured to transmit images of the internal organ to the display device in real time. Further, the disposable flexible tube is connectable to the optical tube and at least partially insertable into the internal organ along with the optical tube. The disposable flexible tube is configured with a second profile on a portion of its outer surface, complementary to the first profile and adapted to engage with the first profile, for coupling the disposable flexible tube with the optical tube. The second profile is a curved profile extending inwardly towards an internal axis of the disposable flexible tube. The curved profile is configured to latch onto the arcuate profile for coupling the disposable flexible tube with the optical tube. The disposable flexible tube also includes one or more channels for administering a medical treatment to the subject. The one or more channels includes a suction channel, an air insufflation channel, a water insufflation channel and a biopsy channel. The suction channel is connectable to a suction device and configured to induce suction at the distal end, wherein the suction channel is configured to collect fluid settled on the distal end during insertion of the endoscope device into the internal organ. The water insufflation channel is connectable to a water container for supplying water to the distal end and is configured to clean the fluids settled on at least one guide lens during insertion of the endoscope device into the internal organ. The air insufflation channel is connectable to an air pump and configured to supply air into the internal organ for insufflation. The biopsy channel is configured to receive an operative instrument and therapeutic fluids for administering medical treatment to the subject. Further, the one or more channels are configured with a port, which is configured to be selectively controlled by the user via the joystick for controlling the operations of each of the one or more channels.

### BRIEF DESCRIPTION OF THE FIGURES

The following detailed description of illustrative embodiments is better understood when read in conjunction with the appended drawings. For the purpose of illustrating the present disclosure, exemplary constructions of the disclosure are shown in the drawings. However, the present disclosure is not limited to a specific device or a tool and instrumentalities disclosed herein. Moreover, those in the art will understand that the drawings are not to scale. Wherever possible, like elements have been indicated by identical numbers:

FIG. 1 is a schematic view of an endoscope device inserted into a subject, in accordance with an exemplary embodiment of the present disclosure;

FIG. 2 is the endoscope device illustrating an optical tube and a disposable flexible tube coupled to each other, in accordance with an exemplary embodiment of the present disclosure;

FIG. 3 is a schematic view of the endoscope device illustrating one or more channels in the flexible tube connected to a system for administering medical treatment to the subject, in accordance with an embodiment of the present disclosure;

FIG. 4 is the endoscope device illustrating connection between a joystick and a first end of the disposable flexible tube, in accordance with an embodiment of the present disclosure;

FIG. 5 is an exploded view of an end of the endo scope device illustrating profiles of the optical tube and the disposable flexible tube coupled to each other, in accordance with an exemplary embodiment of the present disclosure;

FIG. 6 is an exploded view of the end of the endoscope device illustrating profiles of the optical tube and the disposable flexible tube for coupling, in accordance with another embodiment of the present disclosure; and

FIG. 7 is an exploded view of the endoscope device illustrating profiles of the optical tube and the disposable flexible tube for coupling, in accordance with another embodiment of the present disclosure;

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Reference in this specification to “one embodiment” or “an embodiment” means that a particular feature, structure, or characteristic described in connection with the embodiment is included in at least one embodiment of the present disclosure. The appearance of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon, the present disclosure.

### Overview

Various embodiments of the present disclosure provide an endoscope device. The device includes an optical tube and a disposable flexible tube, which are coupled to each other.

The optical tube is an elongated tubular member including a proximal end connectable to a light source and a distal end insertable into an internal organ of a subject via a cavity. The optical tube is configured to be a closed tube and includes at least one light-transmitting element for transmitting light from the proximal end to the distal end for illuminating the internal organ. The distal end is connected to a joystick via a connecting mechanism, which facilitates the user to navigate the distal end within the internal organ of the subject. The distal end also includes an image sensor communicably coupled to a display device. The image sensor is configured to transmit images of the internal organ to the display device in real-time, so that the user can diagnose the internal organ. The optical tube also includes a peripheral surface, wherein a portion of the peripheral surface is configured with a first profile along a longitudinal direction. The first profile may be an arcuate profile extending outwardly from an axis of the optical tube.

The disposable flexible tube includes a first end connectable to the distal end and a second end connectable at a vicinity of the proximal end. The first end being connected to the distal end is also insertable into the internal organ along with the optical tube. The flexible tube includes one or more channels configured for administering a medical treatment to the internal organ, while also facilitating cleaning of biological debris on the distal end accumulated during insertion of the device or during diagnosis of the internal organ. The medical treatment may be one of administering therapeutic fluids, inserting an operative or a surgical instrument and the like for treatment. The one or more channels may be an air insufflation channel, a water insufflation channel, a suction channel and a biopsy channel. Further, the optical tube also includes an outer surface configured with a second profile. The second profile may be a curved profile extending inwardly towards an internal axis of the flexible tube, resembling an annular stud. The first profile and the second profile are configured to engage with each other, for facilitating coupling of the optical tube and the flexible tube. The coupling forms a unitary structure or a seamless construction of the optical tube and the flexible tube. This configuration of the endoscope device ensures that the flexible tube is detachable and disposed-off after use, thereby mitigating the need for cumbersome sterilization process for the flexible tube for reuse. Moreover, it is effortless to sterilize the optical tube or achieve high-level disinfection as it is a closed tube and thus, a new flexible tube may be attached to the optical tube for reuse on another subject.

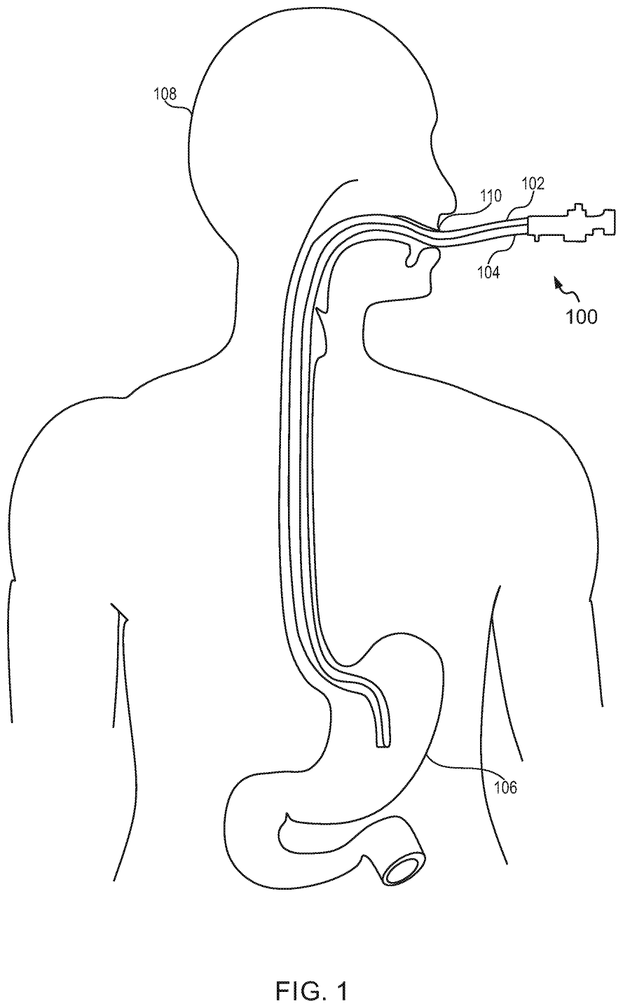

Various embodiments of an endoscope device are explained below in a detailed matter, herein with reference to FIG. 1 to FIG. 7.

FIG. 1, in one exemplary embodiment of the present disclosure, illustrates an endoscope device 100. The device 100 is insertable into an internal organ 106 of a subject 108 for diagnosis via a cavity 110. As an example, the internal organ 106 may be a stomach of the subject 108, and cavity 110 through which the device 100 accesses the stomach may be a mouth of the subject 108. The configuration of the device 100 may alter based on the internal organ 106 to be diagnosed. As such, for diagnosing the internal organ 106 such as an esophagus and a duodenum (not shown in Figures), the device 100 may be configured to be an upper endoscope device. Similarly, the device 100 may be configured to be a colonoscope for diagnosing a colon of the subject 108 (not shown in Figures) and the like. The configuration of the device 100 may also alter based on the size of the cavity 110, through which the device 100 is required to maneuver for entering the internal organ 106. In one implementation, a medical practitioner may induce a cavity 110 at required locations on the subject 108 for accessing and diagnosing the internal organ 106.

The device 100 includes an optical tube 102 and a disposable flexible tube 104, coupled to each other. The optical tube 102 and the flexible tube 104 are coupled to each other due to configuration of their peripheral surfaces, which will be described in detail in subsequent sections of the present disclosure. This configuration of the device 100 ensures that the flexible tube 104 is detached and disposed-off after use, thereby mitigating the need for cumbersome sterilization process of the device 100 for reuse.

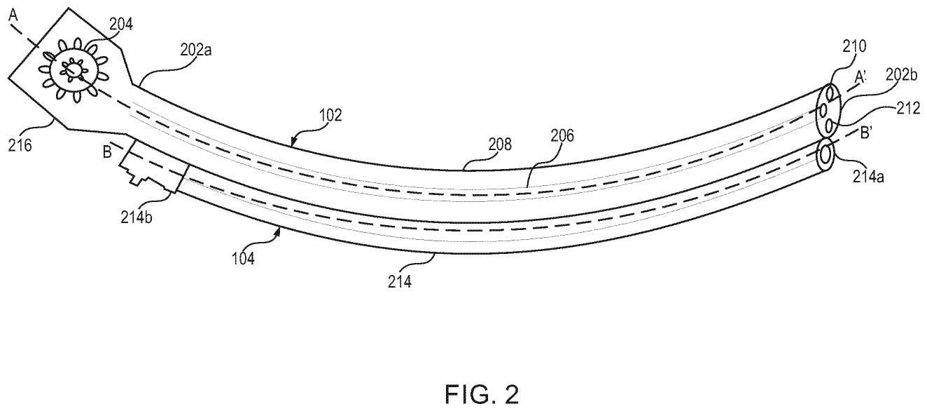

Referring to FIG. 2 in conjunction with FIG. 1, the optical tube 102 is an elongated tubular member extending along a longitudinal direction. The optical tube 102 includes a proximal end 202a and a distal end 202b. The dimensions of the optical tube 102 is configured based on the internal organ 106 to be diagnosed and/or the cavity 110 through which the device 100 is required to be inserted. The proximal end 202a is typically connected to a light source 204, while the distal end 202b is insertable into the subject 108 via the cavity 110 for diagnosing the internal organ 106. The optical tube 102 is configured to transmit light from the light source 204 to the distal end 202b for illuminating the internal organ 106 for diagnosis. The light source 204 for the device 100 may be selected from one of conventional light sources such as but limited to Light Emitting Diodes (LEDs), halogens, xenons and the like. Typically, for facilitating optimal transmission of light from the light source 204, the optical tube 102 is a closed tube which may be configured with a peripheral surface 208 made of a transparent material or an opaque material. The closed tube configuration ensures that the bacterial debris does not enter the optical tube 102 during use. This configuration prevents formation of lumps or clogs of bacterial debris within the optical tube 102 which may obstruct vision of the internal organ 106 for the medical practitioner during diagnosis. The optical tube 102 includes at least one light-transmitting element 206 for optimal transmission of light. The light-transmitting element 206 may be a light-transmitting fiber which includes a glass core (not shown in Figures), for facilitating light transmission by total internal reflection of light within, thereby ensuring optimal transmission of light. In one implementation, the light source 204 may be included in a joystick 216 of the device 100 and thus, the proximal end 202a is directly connected to the joystick 216 for transmitting light. The joystick 216 may also be connected to suitable computing devices or electronics for operating the device 100 via wired or wireless means as per requirement.

The optical tube 102 is also configured with at least one guide lens 210 at the distal end 202b and adjacent to the light-transmitting element 206 of the optical tube 102. The at least one guide lens 210 improves the vision of the medical practitioner on the area of the internal organ 106 at which the light is illuminated. The guide lens 210 may be connected to an eye piece (not shown in the Figures) that may extend from the joystick 216, to enable the medical practitioner to directly view the area of the internal organ 106 under illumination. The guide lens 210 may be selected to be one of a concave lens, a convex lens or any other configuration of lens as per design feasibility and requirement. In one implementation, the light transmitting element 206 may be a bundle coupled to the guide lens 210 (not shown in Figures) for transmitting the captured image. An image sensor 212 is also configured at the distal end 202b for capturing images of the area under illumination. The image sensor 212 may be communicably coupled to a display device 312 for displaying the captured images in real-time. The image sensor 212 may be coupled to the display device 312 by a wired connection (not shown in Figures), which may extend from the joystick 216. In another configuration, the image sensor 212 may be coupled to the display device 312 via a wireless connection, via a wireless module (not shown in Figures) configured in the joystick 216. In another implementation, the image sensor 212 may transmit the captured image via a bundle of fiber via a wired means or wireless means or any other means as per feasibility and requirement. In an embodiment, the joystick 216 may include a control knob (not shown in Figures) for controlling the light intensity or color or properties of the light transmitted through the element 206 for varying the image quality. The control knob may also be operated suitably for freezing the image captures by the image sensor 212 for examination or may operated suitably for recording video of the endoscope procedure.

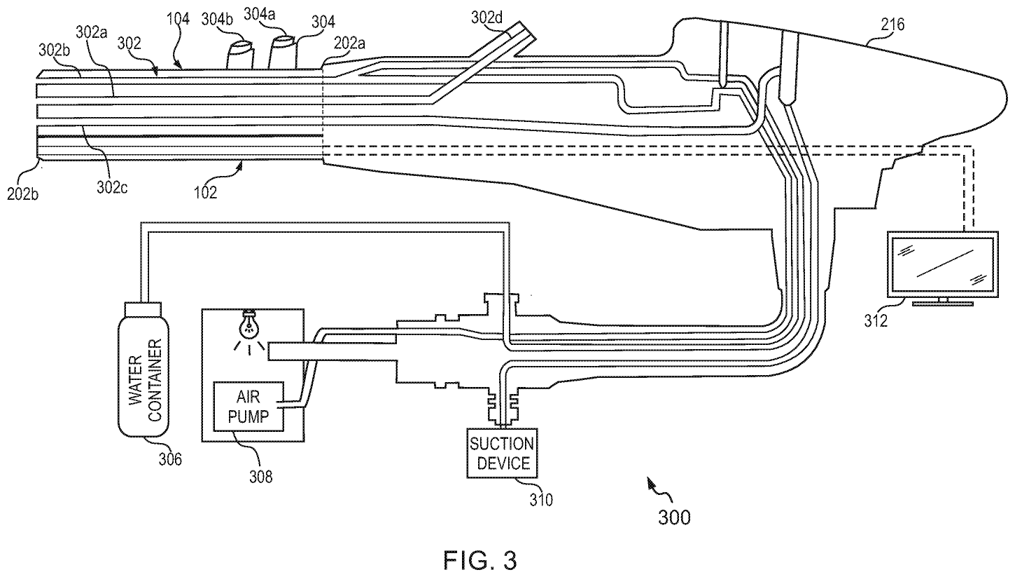

The disposable flexible tube 104 coupled to the optical tube 102 is also an elongated tubular member, wherein its outer surface 214 extends longitudinally about its internal axis B-B′. The flexible tube 104 is also insertable into the internal organ 106 along with the optical tube 102. The flexible tube 104 is configured to facilitate the medical practitioner to administer a medical treatment to the internal organ 106. The flexible tube 104 includes a first end 214a connectable to the distal end 202b and a second end 214b connectable to a vicinity of the proximal end 202a. In one configuration, the second end 214b of the flexible tube 104 is connected to the proximal end 202a. As such, the second end 214b and the proximal end 202a are jointly connected to the joystick 216. This configuration ensures that the joystick 216 can control operations of the optical tube 102 and the flexible tube 104 simultaneously. A plurality of control knobs 304 (for e.g. as shown in FIG. 3) may be provided to the flexible tube 104, which upon actuation by the medical practitioner control operations of the optical tube 102 and the flexible tube 104. Each of the plurality of control knobs 304 may be configured with a suitable valve mechanism (not shown in Figures) for regulating the flow of fluids, air and therapeutic agents therein. In one configuration, length of the flexible tube 104 is equivalent to the optical tube 102. In another configuration, length of the flexible tube 104 is less than the length of the optical tube 102. In another configuration, length of the flexible tube 104 may be longer than the length of the optical tube 102. In one implementation, the joystick 216 includes the plurality of control knobs 304 (for e.g. as shown in FIG. 3), which upon actuation by the medical practitioner control operations of the optical tube 102 and the flexible tube 104.

FIG. 3 in one exemplary embodiment of the present disclosure illustrates a system 300, which includes external components connected to one or more channels 302 of the flexible tube 102 for administering medical treatment to the subject 108. The system 300 may be conventional set-up of devices utilized for performing the endoscopic procedure on the subject 108. The one or more channels 302 are configured to either route fluids (for e.g. medicinal or therapeutic fluids) or an operative instrument (for e.g. a surgical device or a biopsy device). The one or more channels 302 typically includes a suction channel 302a, a water insufflation channel 302b, an air insufflation channel 302c and a biopsy channel 302d. The one or more channels 302 may be configured separately or in combination as per requirement.

The suction channel 302a is connected to a suction device 310 located external to the device 100 and is configured to induce suction at the first end 214a. The suction induced at the first end 214a in turn induces suction at the distal end 202b of the optical tube 102. This configuration clears the fluids that settle and clog the guide lens 210 on the distal end 202b, thereby ensuring unobstructed vision to the medical practitioner during diagnosis. A control knob 304a located on the joystick 216 controls the operation of the suction device 310. The medical practitioner can therefore actuate the knob 304a when the vision is obstructed by the fluids.

The water insufflation channel 302b is connected to a water container 306 located external to the device 100 and is configured to spray water through the first end 214a during insertion of the device 100 into the internal organ 106 via the cavity 110. The water insufflation may lead to reduced post-procedural pain to the subject 108, improved recovery rate and discomfort during the procedure. A control knob 304b located on the joystick 216 controls the operation of the water insufflation into the subject 108, as and when required. In another implementation, the water insufflation may also be controlled via a foot pedal (not shown in Figures) coupled to the water container 306 via an external pump. Further, the air insufflation channel 302c is connected to an air pump 308 located external to the device 100 and is configured to blow air through the first end 214a into the internal organ 106 upon insertion of the device 100. The air insufflation may enhance visual inspection of the medical practitioner during diagnosis. The control knob 304b located on the joystick 216 may be linked to the air insufflation channel 302c for controlling the operation of the air insufflation into the subject 108 as and when required.

The biopsy channel 302d may be located at a central portion of the joystick 216. The biopsy channel 302d may be dimensioned for receiving and directing the operative instrument into the internal organ 106 via the first end 214a. The operative instrument may be a tissue collecting instrument or a surgical instrument or any other instrument as per feasibility and requirement. Further, the biopsy channel 302d may also be connectable to a fluid container for administering a fluid into the internal organ 106.

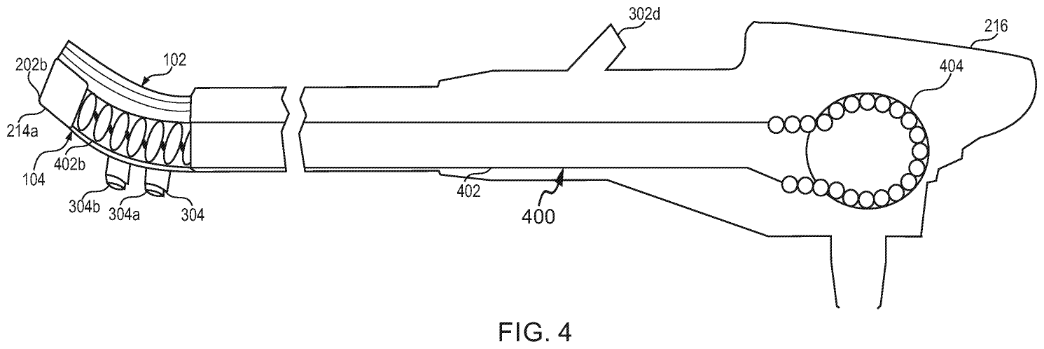

FIG. 4 in one exemplary embodiment of the present disclosure illustrates a connecting mechanism 400 of the joystick 216 coupled to the first end 214a of the flexible tube 104. The connecting mechanism 400 may integrate with the flexible tube 104 upon establishing its connection with the joystick 216. The joystick 216 is configured to maneuver the first end 214a and hence the distal end 202b via the connecting mechanism 400. This configuration ensures that the medical practitioner can maneuver the device 100 for accessing intricate location within the internal organ 106, thereby ensuring thorough diagnostics. In another configuration, the connecting mechanism 400 may be configured to the optical tube 102 to facilitate maneuvering.

The connecting mechanism 400 may be a wire-pulley mechanism. The wire-pulley mechanism includes a wire 402 configured to loop about the joystick 216 and the first end 214a. The wire 402 has one end 402a looped about a plurality of pulleys 404 in the joystick 216 and another end 402b connected to the first end 214a. The plurality of pulleys 404 are coupled to an actuator (not shown in Figures), which operates upon actuation of a button (not shown in Figures). The actuator is configured to operate the wire 402 in a push-pull manner, so that the first end 214a can bend towards its left side or the right side as per requirement. Based on these movements, the medical practitioner can maneuver the device 100 suitably within the internal organ 106. In another configuration, the connecting mechanism 400 may be a mechanism other than the wire-pulley mechanism as per design feasibility and requirement.

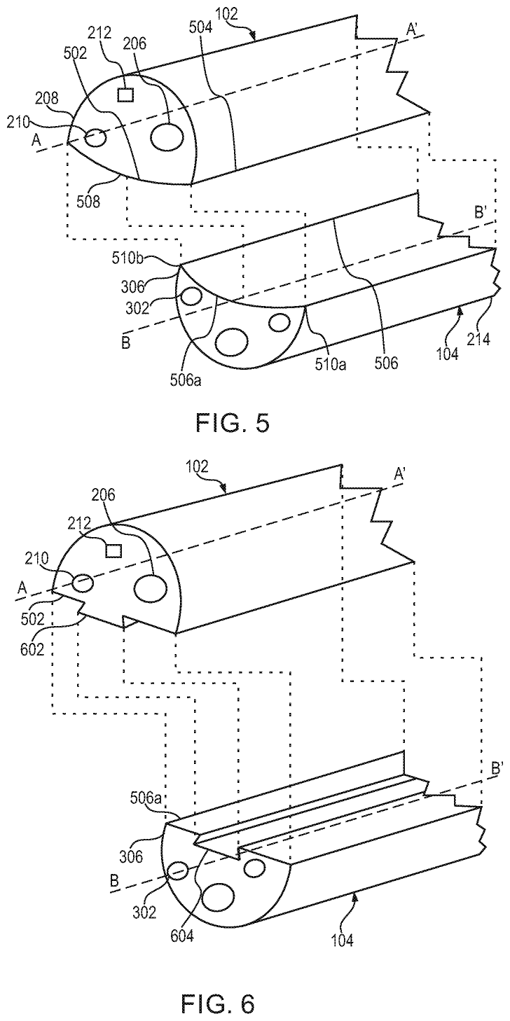

FIG. 5 in one exemplary embodiment of the present disclosure illustrates coupling of the optical tube 102 and the flexible tube 104. The optical tube 102 includes the peripheral surface 208 extending in the longitudinal direction. The peripheral surface 208 is configured with a lateral surface 502, which engages with the flexible tube 104 for coupling. A portion of the peripheral surface 208 is configured with a first profile 504, which also extends along the longitudinal direction. The portion of the peripheral surface 208 including the first profile 504 may be defined on the lateral surface 502. The first profile 504 may be an arcuate profile extending outwardly from an axis A-A′ of the optical tube 102. This configuration of the first profile 504 resembles the sector of the circle (in a cross-sectional view) or a protrusion 508, due to its extension about the perimeter of the lateral surface 502b in the arcuate manner. Complimentarily, the flexible tube 104 is also configured with an outer surface 214 extending along its internal axis B-B′. The outer surface 214 includes an engaging surface 506a which is configured to engage with the first profile 504 or the lateral surface 502b for coupling the flexible tube 104 with the optical tube 102. A portion of the outer surface 214 is also configured with a second profile 506 which is complementary in configuration to that of the first profile 504 for facilitating coupling. The second profile 506 may be an arcuate profile projecting inwardly to the internal axis B-B′ of the flexible tube 104. This configuration of the second profile 506 may resemble an annular stud 510 having a recessed central portion 510a and a raised end portion 510b. The annular stud 510 engages with the protrusion 508, for coupling the optical tube 102 with the flexible tube 104. In this configuration, the tolerance of the annular stud 510 with respect to the protrusion 508 is minimal, and thereby establishing an interference fit with the protrusion for coupling the optical tube 102 with the flexible tube 104. The coupling of the optical tube 102 and the flexible tube 104 results in a cylindrical tube of seamless construction, thereby preventing any discomfort to the subject 108 during use. In one implementation, the optical tube 102 and the flexible tube 104 couple to each other via a snap-fit configuration.

In one configuration, the lateral surface 502 of the optical tube 102 in combination with the peripheral surface 208 conform to a cross-section which resembles a sector of a circle. In another implementation, the lateral surface 502 in combination with the peripheral surface 208 may conform to other geometric cross-sections as per feasibility and requirement.

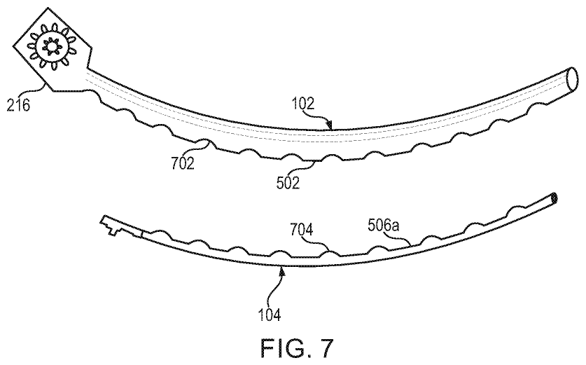

In one configuration, the first profile 504 may be a notch member projecting inwardly or extending outwardly as per design feasibility and requirement. In another configuration, the first profile 504 may be a dovetail protrusion 602 (as shown in FIG. 6) extending outwardly from the axis A-A′. The first profile 504 may also be configured along the entire length of the optical tube 102 (for e.g. as shown in FIGS. 5 and 6) or may be configured intermittently about the length of the optical tube 102 (for e.g. as shown in FIG. 7). This versatility enables to manufacture or select the required type of engagement of the flexible tube 104 as per requirement. Moreover, the first profile 504 which extends outwardly from the axis A-A′ acts as a male type of connector, while the first profile 504 which projects inwardly towards the axis A-A′ acts as a female type of connector.

In one configuration, the portion of the outer surface 214 with the second profile 506 may be the engaging surface 506a of the flexible tube 104. The second profile 506 may extend along the internal axis B-B′ of the flexible tube 104 or may be configured intermittently about the length of the flexible tube 104. Similar to configuration of the optical tube 102, the combination of the engaging surface 506a and the outer surface 214 resembles a recessed section of the circle. In another implementation, the outer surface 214 and the engaging surface 506a may conform to other geometric cross-sections as per feasibility and requirement.

In an embodiment, the first profile 504 and the second profile 506 may be configured such that the structural integrity of either of the optical tube 102 and the flexible tube 104 is maintained.

In another embodiment, the first profile 504 and the second profile 506 may be configured with a thread profile,

In an embodiment, the axis A-A′ and the internal axis B-B′ may be a central axis of the optical tube 102 and the flexible tube 104, respectively. In another configuration, the axis A-A′ and the internal axis B-B′ may be any axis defined on the optical tube 102 and the flexible tube 104 for configuring the first profile 504 and the second profile 506.

In one configuration, the second profile 506 may be a notch member projecting inwardly or extending outwardly as per design feasibility and requirement. In another configuration, the second profile 506 may be a dovetail groove 604 (as shown in FIG. 6) projecting inwardly from the internal axis B-B′. The second profile 506 may also be configured along the entire length of the flexible tube 104 (for e.g. as shown in FIGS. 5 and 6) or may be configured intermittently about the length of the flexible tube 104 (for e.g. as shown in FIG. 7). This versatility enables to manufacture or select the required type of engagement of the flexible tube 104 as per requirement. Moreover, the second profile 506 which extends outwardly from the internal axis B-B′ acts as a male type of connector, while the first profile 504 which projects inwardly towards the internal axis B-B′ acts as a female type of connector.

Referring to FIG. 6, the first profile 504 is the dovetail protrusion 602 and the second profile 506 is the dovetail groove 604. In this configuration, the first profile 504 i.e. the optical tube 102 is slidably inserted within the second profile 506 along the entire length. A connector (not shown in Figures) may be provided at the distal end 202b for locking the flexible tube 104, when the first end 214a meets the distal end 202b. Similar configuration may be provided towards the proximal end 202a for locking the flexible tube 104. The connector may be any one of a mechanical connector such as a strap mechanism, a thread mechanism and the like, or a magnetic connector or any other connector which serves the purpose of locking the flexible tube 104 upon coupling. This configuration ensures that the flexible tube 104 remains snuggly fitted to the optical tube 102 during use. In another configuration, the first profile 504 and the second profile 506 may be configured with minimal tolerance for establishing an interference fit there between.

FIG. 7 illustrates the first profile 504 being a notch member 702 which is intermittently configured along the length of the optical tube 102. Complimentarily, the second profile 506 is a protruded member 704 also configured intermittently along the length of the flexible tube 104. The distance between each of the protruded member 704 may be configured based on design feasibility and requirement. In this configuration, the medical practitioner inserts each of the protruded members 704 with its corresponding notch members 702 on the optical tube 102 and press fitted to form the seamless construction. For establishing interference fit between the protruded members 704 and the notch members 702, a minimum tolerance is maintained. In one configuration, the dimensions of the notch members 702 and the protruded members 704 may be selected without compromising structural integrity of the optical tube 102.

Thus, the coupling of the first profile 504 and the second profile 506 forms the unitary structure or the seamless construction of the optical tube 102 and the flexible tube 104, so that there is minimal discomfort to the subject 108 during use. This configuration of the endoscope device 100 ensures that the flexible tube 104 is detached and disposed-off after use, thereby mitigating the need for cumbersome sterilization process for the flexible tube 104 for reuse. Moreover, it is effortless to sterilize the optical tube 102 as it is a closed tube and thus, a new flexible tube 104 may be attached to the optical tube 102 for reuse on another subject 108.

The benefits and advantages described above may relate to one embodiment or may relate to several embodiments. The embodiments are not limited to those that solve any or all of the stated problems or those that have any or all of the stated benefits and advantages.

The above description is given by way of example only and various modifications may be made by those skilled in the art. The above specification, examples, and data provide a complete description of the structure and use of exemplary embodiments. Although various embodiments have been described above with a certain degree of particularity, or with reference to one or more individual embodiments, those skilled in the art could make numerous alterations to the disclosed embodiments without departing from the spirit or scope of this specification.

What is claimed is:

1. An endoscope device, comprising:
an optical tube including a proximal end connectable to a light source via a joystick and a distal end insertable into an internal organ of a subject via a cavity, the optical tube configured to be a closed tube and includes at least one light-transmitting element for transmitting light from the proximal end to the distal end, wherein a portion of a peripheral surface of the optical tube is configured with a first profile along a longitudinal direction, the first profile configured to be an arcuate profile extending outwardly from an axis of the optical tube;
at least one guide lens configured at the distal end of the optical tube and located adjacent to the light-transmitting element for improving illumination;
an image sensor positioned to the distal end of the optical tube and communicably coupled to a display device, the image sensor configured to transmit images of the internal organ of the subject to the display device in real time; and
a disposable flexible tube connectable to the optical tube and at least partially insertable into the internal organ along with the optical tube, the disposable flexible tube configured with a second profile on a portion of its outer surface, complementary to the first profile and adapted to engage with the first profile, for coupling the disposable flexible tube with the optical tube, wherein the second profile is a curved profile extending inwardly towards an internal axis of the disposable flexible tube, the curved profile configured to latch onto the arcuate profile for coupling the disposable flexible tube with the optical tube
wherein, the disposable flexible tube includes one or more channels for administering a medical treatment to the subject, the one or more channels includes:
a suction channel connectable to a suction device configured to induce suction at the distal end, the suction channel configured to collect fluid settled on the distal end during insertion of the endoscope device into the internal organ,
a water insufflation channel connectable to a water container for supplying water to the distal end, the water insufflation channel configured to clean the fluids settled on at least one guide lens during insertion of the endoscope device into the internal organ,
an air insufflation channel connectable to an air pump and configured to supply air into the internal organ for insufflation, and
a biopsy channel configured to receive an operative instrument and therapeutic fluids for administering medical treatment to the subject,
the one or more channels are configured with a port, the port configured to be selectively controlled by the user via the joystick for selectively controlling the operations of each of the one or more channels,
wherein, the optical tube and the flexible tube upon coupling is configured to form a cylindrical tube of seamless construction.
